(19) United States
(12) Patent Application Publication
(10) Pub. No.: US 2010/0076687 A1
(43) Pub. Date: Mar. 25, 2010

(54) DOCUMENT MANAGEMENT ENVIRONMENTAL FOOTPRINT CALCULATOR

(75) Inventors: Victoria Fornaro DeYoung, Webster, NY (US); Patricia Anne Calkins-Martin, Webster, NY (US)

Correspondence Address: PEPPER HAMILTON LLP, 500 GRANT STREET, ONE MELLON CENTER, 50TH FLOOR, PITTSBURGH, PA, 15219, US

(73) Assignee: Xerox Corporation, Norwalk, CT (US)

(21) Appl. No.: 12/238,063

(22) Filed: Sep. 25, 2008

(51) Int. Cl. G01N 33/00

(52) U.S. Cl. 702/2

(57) ABSTRACT

Systems and methods for measuring an environmental footprint for a printing device. A system for measuring an environmental footprint for a printing device may include a processor communicating with an output device and a computer-readable storage medium in communication with the processor. The computer-readable storage medium may contain one or more programming instructions for providing at least one value for a measurement factor pertaining to the printing device, selecting one or more environmental footprint outputs based on a total impact of creation, usage and disposal of the printing device, determining an estimated emissions profile regarding one or more selected environmental footprint outputs including an amount of emissions to air and/or emissions to water, and causing a difference between the estimated emissions profile and an initial emissions profile to be displayed on the output device.

### CROSS REFERENCE TO RELATED APPLICATIONS

This application is related to U.S. patent application Ser. No. 12/237,974 filed Sep. 25, 2008.

### BACKGROUND

1. Technical Field

This application relates to environmental footprint calculations in a document management process. More specifically, the present disclosure relates to systems and methods of providing and quantifying information to create relative comparisons regarding dimensions of environmental performance for sets of office products.

2. Description of Related Prior Art

Global awareness has led to increased concern regarding the amount of carbon or equivalent emissions being released into the atmosphere. A measurement of these emissions is generally referred to as a carbon footprint. Many entities are interested in calculating their carbon footprint. Such calculations can assist with determining methods of reducing associated emissions. For example, factors can be monitored such as gas usage, electricity usage and mileage associated with travel to calculate a carbon footprint. Carbon footprints can be calculated for a personal household, an international corporation, or any other entity.

Within a business environment, office products tend to consume large amounts of energy and greatly contribute to an entity's carbon footprint. For example, office products can include fleets of equipment such as copiers, printers and multi-function devices. Entities may track the carbon footprint for office products via a carbon calculator that tracks, for example, greenhouse emissions. Conventional calculators have limited functionality, however, because they are based solely on power usage in connection with the office products.

### SUMMARY

Before the present systems, devices and methods are described, it is to be understood that this disclosure is not limited to the particular systems, devices and methods described, as these may vary. It is also to be understood that the terminology used in the description is for the purpose of describing the particular versions or embodiments only, and is not intended to limit the scope.

It must also be noted that as used herein and in the appended claims, the singular forms “a,” “an,” and “the” include plural references unless the context clearly dictates otherwise. Thus, for example, reference to a “device” is a reference to one or more devices and equivalents thereof known to those skilled in the art, and so forth. Unless defined otherwise, all technical and scientific terms used herein have the same meanings as commonly understood by one of ordinary skill in the art. Although any methods, materials, and devices similar or equivalent to those described herein can be used in the practice or testing of embodiments, the preferred methods, materials, and devices are now described. All publications mentioned herein are incorporated by reference. Nothing herein is to be construed as an admission that the embodiments described herein are not entitled to antedate such disclosure by virtue of prior invention. As used herein, the term “comprising” means “including, but not limited to.”

In an embodiment, a system for measuring an environmental footprint for a printing device may include a processor communicating with an output device and a computer-readable storage medium in communication with the processor. The computer-readable storage medium may contain one or more programming instructions for providing at least one value for a measurement factor pertaining to the printing device, selecting one or more environmental footprint outputs based on a total impact of creation, usage and disposal of the printing device, determining an estimated emissions profile regarding one or more selected environmental footprint outputs including an amount of emissions to air and/or emissions to water, and causing a difference between the estimated emissions profile and an initial emissions profile to be displayed on the output device.

In an embodiment, a method of automatically measuring an expected environmental performance for a printing device may include providing a first value for a measurement factor in connection with a baseline analysis for the printing device, providing a second value for a measurement factor in connection with a scenario analysis for the printing device, selecting the one or more dimensions of environmental performance, determining an estimated emissions profile regarding the one or more selected dimensions of environmental performance including emissions to air and/or emissions to water, producing a first output from the estimated emissions profile based on a total impact of creation, usage and disposal of the printing device associated with the one or more selected dimensions of environmental performance in connection with the first value, producing a second output from the estimated emissions profile based on a total impact of creation, usage and disposal of the printing device associated with the one or more selected dimensions of environmental performance in connection with the second value, displaying the first and second outputs, determining a difference of the first and second outputs, and displaying the difference.

In an embodiment, a method of quantifying an environmental footprint for a group of office products may include providing a value for a measurement factor in connection with a baseline analysis, selecting one or more dimensions of environmental performance based on a total impact of creation, usage and disposal of the group of office products, determining an output based on the total impact of creation, usage and disposal of the group of office products associated with the one or more dimensions of environmental performance from the value including an amount of emissions to air and/or emissions to water, making one or more adjustments to a document management process, identifying one or more changes to the output resulting from the one or more adjustments made to the document management process, and performing an action responsive to both the making one or more adjustments to the document management process and the identifying one or more changes to the output.

### BRIEF DESCRIPTION OF THE DRAWINGS

Aspects, features, benefits and advantages of the present application will be apparent with regard to the following description and accompanying drawings, of which:

FIGS. 1-2 depict flow diagrams of exemplary methods of providing and quantifying information regarding dimensions of environmental performance for various sets of office products according to embodiments.

FIG. 3 is a block diagram of exemplary internal hardware that may be used to contain or implement program instructions according to an embodiment.

### DETAILED DESCRIPTION

The following terms shall have, for the purposes of this application, the meanings set forth below.

For the purposes of the discussion below, “office products” refers to mechanical instrumentation configured to provide desired functions for use in an office setting or the like. For example, the office product may provide a printing function. An office product may additionally or alternatively provide a copying function. Further, an office product may perform multiple functions such as, for example, copying, printing and faxing functions. Additional types of office products may also be included within the scope of this disclosure.

A “printing device” is an electronic device that is capable of receiving commands and printing text and/or images on a substrate and/or scanning a document. Printing devices may include, but are not limited to, network printers, production printers, copiers, facsimile devices, other devices using ink or toner, and scanners. Printing devices may also perform a combination of functions such as printing/scanning/copying/faxing, in which case such devices may be considered to be multifunctional devices.

An “environmental footprint” refers to an output or dimension of environmental performance utilized to monitor the release of carbon or other emissions due to, for example, business operations. For example, the environmental footprint for a set of office products may be realized by monitoring use and activity of involved equipment during an inventory life cycle. Additional factors pertaining to realization of an environmental footprint may also be included within the scope of this disclosure.

A “baseline analysis” refers to an analysis of a current setup. For example, a baseline analysis may include calculations of dimensions of environmental performance for a set of office products already in use.

A “scenario analysis” refers to an analysis of a prospective setup. For example, a scenario analysis may include calculations of dimensions of environmental performance for a set of office products as the products may be used.

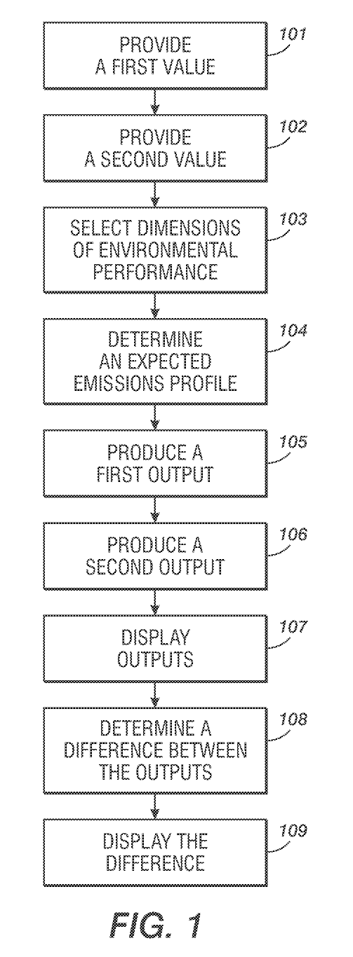

FIG. 1 depicts a flow diagram of a first exemplary method of automatically measuring an expected environmental performance for a printing device according to an embodiment. As shown in FIG. 1, a first value for a measurement factor in connection with a baseline analysis for the printing device may be provided 101. The first value may pertain to a baseline analysis and may include, for example, a machine weight. Alternatively, the first value may include a speedband value for at least a portion of the first set from which an average machine weight is determined. The first value may alternatively include the number of images printed, information as to a cartridge recycling process or the lack thereof, a before and after duplex rate, and a plurality of different types of paper and recycle content information, and/or the like.

Additionally, a second value for the measurement factor may be provided 102 in connection with a scenario analysis for the printing device. The second value may include, for example, a machine weight. Alternately, the second value may include a speedband value for at least a portion of the second set from which an average machine weight is determined. The second value may alternatively include the number of images printed, information as to a cartridge recycling process or lack thereof, a before and after duplex rate, and a plurality of different types of paper and recycle content information, and/or the like.

One or more dimensions of environmental performance may be selected 103. Regarding the one or more selected dimensions of environmental performance 103, an estimated emissions profile may be determined 104. The estimated emissions profile may include, for example, emissions to air and emissions to water. In an embodiment, the estimated emissions profile may include paper use. In another embodiment, determining the estimated emissions profile regarding the one or more selected dimensions of environmental performance may include determining the emissions of, for example, carbon dioxide, methane and/or nitrous oxide. Additional and/or alternate dimensions may also be used within the scope of this disclosure.

A first output may be produced 105 from the estimated emissions profile. The first output may be based on a total environmental impact of creation, usage and disposal of the printing device associated with the one or more selected dimensions of environmental performance 103 in connection with the first value. Likewise, a second output may be produced 106 from the estimated emissions profile. The second output may be based on a total environmental impact of creation, usage and disposal of the printing device associated with the one or more dimensions of environmental performance 103 in connection with the second value. Additional and/or alternate factors contributing to the production of the first and/or second output may be used within the scope of this disclosure.

The first and second outputs may be displayed 107. A difference of the first and second outputs may be determined 108. The determined difference 108 may be displayed 109.

In an embodiment, the first output may be compared to the second output. Alternatively, an analysis may be performed for the one or more selected dimensions of environmental performance 103 associated with the first output and the second output. This analysis may determine whether an action based on the second output would best optimize efficiencies and reduce the first output. For example, a report may be produced including one or more graphs and/or charts and/or information regarding the comparison, the outputs and/or an analysis of the outputs. The first output and the second output may be displayed 106. The display may occur, for example, in a graphical form and contain additional information to assist with an analysis.

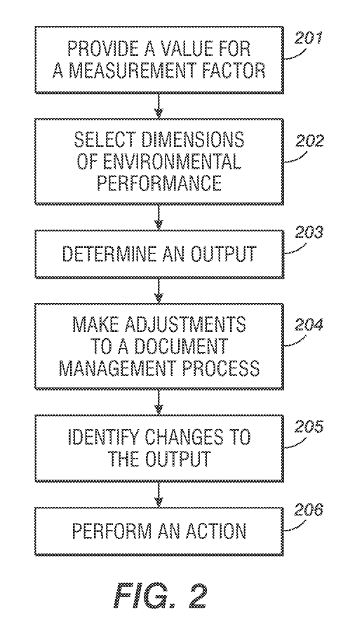

FIG. 2 depicts a flow diagram of a second exemplary method of providing information regarding dimensions of environmental performance for a group of office products according to an embodiment. As shown in FIG. 2, a value for a measurement factor in connection with a baseline analysis may be provided 201. For example, the value may be provided 201 by a user. In an embodiment, the group of office products may include a copying device, a printing device, a multi-function device and/or the like.

One or more dimensions of environmental performance may be selected 202. An output associated with the one or more dimensions of environmental performance from the value may be determined 203. Determining the output may include determining an amount of emissions to air and emissions to water. In an embodiment, determining the output associated with the one or more dimensions of environmental performance may include determining an amount of paper use. In another embodiment, determining 203 the output associated with the one or more dimensions of environmental performance may include determining an amount of carbon dioxide, methane, and/or nitrous oxide emissions. The output may be based on a total impact of creation, usage and disposal for the set of one or more office products with respect to the one or more dimensions of environmental performance. The output may be referred to, for example, as a baseline environmental footprint. Additional and/or alternate factors contributing to the determination 203 of the output may be used within the scope of this disclosure.

One or more adjustments to a document management process may be made 204. One or more changes to the output resulting from the one or more adjustments to the document management process may be identified 205. The one or more adjustments and/or the one or more changes may be displayed to a user. The one or more adjustments to the document management process and the one or more changes to the output may be used to determine one or more actions to be performed 206. The user may perform 206 one or more actions and/or the system may recommend one or more actions. In an embodiment, performing the action may include determining a difference for each of the one or more changes to the output. In another embodiment, performing the action may include displaying a difference for each of the one or more changes to the output.

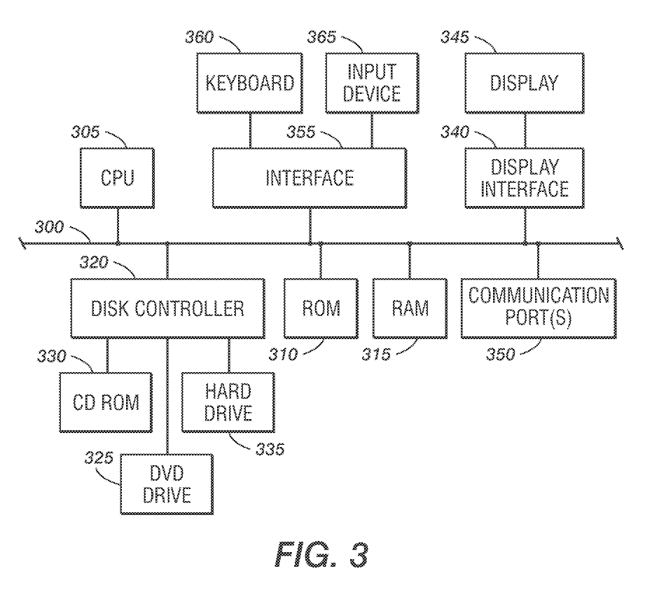

FIG. 3 depicts a block diagram of an exemplary system that may be used to contain or implement program instructions for measuring an environmental footprint of a printing device. Referring to FIG. 3, the exemplary system may include internal hardware which may be used to contain or implement program instructions according to an embodiment. A bus 300 serves as the main inform highway interconnecting the other illustrated components of the hardware. CPU 305 is the central processing unit of the system, performing calculations and logic operations required to execute a program. Read only memory (ROM) 310 and random access memory (RAM) 315 constitute exemplary memory devices or storage media.

An output device may communicate with the processor. Further, a computer-readable storage medium including one or more programming instructions may communicate with the processor. A disk controller 320 interfaces with one or more optional disk drives to the system bus 300. These disk drives may include, for example, external or internal DVD drives 325, CD ROM drives 330 or hard drives 335. As indicated previously, these various disk drives and disk controllers are optional devices.

Programming instructions may be stored in the ROM 310 and/or the RAM 315. Optionally, program instructions may be stored on any other tangible computer-readable storage medium, such as a hard drive, a compact disk, a digital disk, a memory or any other tangible storage medium. The programming instructions may include the provision of at least one value for a measurement factor pertaining to the printing device.

One or more environmental footprint outputs based on a total impact of creation, usage and disposal of the one or more office products may be selected. These outputs may take into consideration a total life cycle perspective of the printing device in regards to creation, usage and disposal of the printing device. An estimated emissions profile may be determined regarding the one or more selected environmental footprint outputs. Determining the estimated emissions profile may include determining an amount of emissions to air and emissions to water. In an embodiment, determining the estimated emissions profile regarding the one or more selected environmental footprint outputs may include determining an amount of paper use. In another embodiment, determining the estimated emissions profile regarding the one or more selected environmental footprint outputs may include determining an amount of carbon dioxide, methane and/or nitrous oxide emissions. A difference between the estimated emissions profile and an initial emissions profile may be caused to be displayed on the output device.

An optional display interface 340 may permit information from the bus 300 to be displayed on a display 345 in audio, graphic or alphanumeric format. Communication with external devices may occur using various communication ports 350. Information detailing the one or more environmental footprint outputs may be displayed on an output device. In an embodiment, displaying such information may include causing the information to be displayed on printing devices, display devices and/or the like. Additional and/or alternate output devices may be utilized within the scope of this disclosure.

In addition to the standard computer-type components, the hardware may also include an interface 355 which allows for receipt of data from input devices such as a keyboard 360 or other input device 365 such as a mouse, remote control, pointer and/or joystick.

An embedding system may optionally be used to perform one, some or all of the operations described herein. Likewise, a multiprocessor system may optionally be used to perform one, some or all of the operations described herein.

It will be appreciated that various of the above-disclosed and other features and functions, or alternatives thereof, may be desirably combined into many other different systems or applications. It will also be appreciated that various presently unforeseen or unanticipated alternatives, modifications, variations or improvements therein may be subsequently made by those skilled in the art which are also intended to be encompassed by the disclosed embodiments.

What is claimed is:

1. A system for measuring an environmental footprint for a printing device, comprising:
a processor in communication with an output device; and
a computer-readable storage medium in communication with the processor, wherein the computer-readable storage medium includes one or more programming instructions for:
providing at least one value for a measurement factor pertaining to the printing device,
selecting one or more environmental footprint outputs based on a total impact of creation, usage and disposal of the printing device,
determining an estimated emissions profile regarding one or more selected environmental footprint outputs comprising an amount of one or more of the following: emissions to air and emissions to water, and
causing a difference between the estimated emissions profile and an initial emissions profile to be displayed on the output device.

2. The system of claim 1, wherein the output device comprises one or more of the following: a printing device and a display device.

3. The system of claim 1, wherein determining the estimated emissions profile regarding the one or more selected environmental footprint outputs further comprises determining an amount of paper use.

4. The system of claim 1, wherein determining the estimated emissions profile regarding the one or more environmental footprint outputs comprises determining an amount of one or more of the following:
carbon dioxide emissions;
methane emissions; and
nitrous oxide emissions.

5. A method of automatically measuring an expected environmental performance for a printing device, comprising:
providing a first value for a measurement factor in connection with a baseline analysis for the printing device;
providing a second value for a measurement factor in connection with a scenario analysis for the printing device;
selecting the one or more dimensions of environmental performance;
determining an estimated emissions profile regarding the one or more selected dimensions of environmental performance comprising one or more of the following: emissions to air and emissions to water;
producing a first output from the estimated emissions profile based on a total impact of creation, usage and disposal of the printing device associated with the one or more selected dimensions of environmental performance in connection with the first value;
producing a second output from the estimated emissions profile based on a total impact of creation, usage and disposal of the printing device associated with the one or more selected dimensions of environmental performance in connection with the second value;
displaying the first and second outputs;
determining a difference between the first and second outputs; and
displaying the difference.

6. The method of claim 5, wherein the printing device is characterized by one or more machine weights and the measurement factor comprises one of the machine weights.

7. The method of claim 5, wherein providing a first value for a measurement factor comprises:
receiving a speedband value; and
determining an average machine weight from the speedband value.

8. The method of claim 5, wherein providing a second value for the measurement factor comprises:
receiving a speedband value; and
determining an average machine weight from the speedband value.

9. The method of claim 5, wherein providing a first value for the measurement factor comprises providing one or more of the following:
a number of images printed;
information as to a cartridge recycling process;
a before and after duplex rate;
paper content information; and
recycle content information.

10. The method of claim 5, wherein providing a second value for the measurement factor comprises providing one or more of the following:
a number of images printed;
information as to a cartridge recycling process;
a before and after duplex rate;
paper content information; and
recycle content information.

11. The method of claim 5, wherein determining the estimated emissions profile regarding the one or more selected dimensions of environmental performance further comprises determining an amount of paper use.

12. The method of claim 5, wherein determining the estimated emissions profile regarding the one or more selected dimensions of environmental performance comprises determining one or more of the following:
carbon dioxide emissions;
methane emissions; and
nitrous oxide emissions.

13. The method of claim 5, further comprising comparing the first output and the second output.

14. The method of claim 5, further comprising providing an analysis of the one or more selected dimensions of environmental performance including the first and second outputs to determine whether an action based on the second output would best optimize efficiencies and reduce the first output.

15. A method of quantifying an environmental footprint for a group of office products, comprising:
providing a value for a measurement factor in connection with a baseline analysis;
selecting one or more dimensions of environmental performance based on a total impact of creation, usage and disposal of the group of office products;
determining an output based on the total impact of creation, usage and disposal of the group of office products associated with the one or more dimensions of environmental performance from the value comprising an amount of one or more of the following: emissions to air and emissions to water;
making one or more adjustments to a document management process;
identifying one or more changes to the output resulting from the one or more adjustments made to the document management process; and
responsive to both the making one or more adjustments to the document management process and the identifying one or more changes to the output, performing an action.

16. The method of claim 15, wherein performing the action comprises determining a difference for each of the one or more changes to the output.

17. The method of claim 15, wherein performing the action comprises displaying a difference for each of the one or more changes to the output.

18. The method of claim 15, wherein determining an output associated with the one or more dimensions of environmental performance further comprises determining an amount of paper use.

19. The method of claim 15, wherein determining an output associated with the one or more dimensions of environmental performance comprises determining an amount of one or more of the following:
carbon dioxide emissions;
methane emissions; and
nitrous oxide emissions.
